(19) United States
(12) Patent Application Publication
(10) Pub. No.: US 2006/0234379 A1
(43) Pub. Date: Oct. 19, 2006

(54) Cell separation method using hydrophobic solid supports

(76) Inventors: Hee-kyun Lim, Suwon-si (KR); Kyu-youn Hwang, Incheon-si (KR); Joon-ho Kim, Seongnam-si (KR); Kak Namkoong, Seoul (KR); Chin-sung Park, Yongin-si (KR)

Correspondence Address: CANTOR COLBURN, LLP, 55 GRIFFIN ROAD SOUTH, BLOOMFIELD, CT, 06002, US

(21) Appl. No.: 11/405,106

(22) Filed: Apr. 17, 2006

(30) Foreign Application Priority Data
Apr. 15, 2005 (KR) 10-2005-0031406
Sep. 5, 2005 (KR) 10-2005-0082442

(51) Int. Cl. C12N 5/02; C12N 5/00

(52) U.S. Cl. 435/378

(57) ABSTRACT

Provided is a method of separating cells using a hydrophobic solid support. The method comprises contacting a solution containing cells with a hydrophobic solid support having a water contact angle between 70 and 90 degrees. By allowing cells to be adsorbed to the hydrophobic solid substrate, the cells can be separated. Thus, the cell separation efficiency can be rapidly and simply increased.

### CROSS-REFERENCE TO RELATED PATENT APPLICATION

This application claims the benefit of Korean Patent Application No. 10-2005-0031406, filed on Apr. 15, 2005 and Korean Patent Application No. 10-2005-0082442, filed on Sep. 5, 2005, in the Korean Intellectual Property Office, the disclosures of which are incorporated herein in their entirety by reference.

### BACKGROUND OF THE INVENTION

1. Field of the Invention

The present invention relates to a cell separation method using hydrophobic solid supports.

2. Description of the Related Art

The separation of cells from mixtures containing them and unwanted impurities is a challenging problem in the art. This is particularly the case where the cells are present in a culture broth, a biological sample or a similar complex mixture, as the methods employed need to allow a high extent of capture of the target cells in an intact state. This means that the reagents used in cell concentration and separation steps must capture the cells very efficiently over a broad range of sample cell densities. Also, the reagents used should not interfere with downstream processes using the cells, for example, the recovery of nucleic acid from the cells and/or the processing of nucleic acid from the cells.

Various methods are known to bind cells to a solid support. In one method, cells with phagocytic activity may be captured by their ability to bind or engulf a particulate solid phase, for example, beads, and thereby be readily separated. In this case, the cell-containing sample needs simply to be contacted or incubated with the solid phase under appropriate conditions. This kind of cell capture is not dependent on specific binding.

In another method, non-specific binding of cells to a solid support may be achieved by appropriate choice of the properties of the solid support and the surrounding conditions, e.g., the chemical or physical nature of the surface of the solid support (e.g., hydrophobicity or charge), the pH or composition of the isolation medium, etc. The nature of the target cells may also play a role. For example, it has been shown that certain hydrophobic cells may be readily bound non-specifically to hydrophobic surfaces, whereas hydrophilic cells may be readily bound to hydrophilic surfaces. Negatively charged cells such as B-lymphocytes have also been observed to have a high degree of non-specific binding to slightly-positively charged surfaces. Thus solid supports having appropriately charged surfaces for binding of a desired cell type may be used. Appropriate buffers may be used as media for cell separation to achieve the conditions appropriate for cell binding by simply placing the solid support and the sample in contact with an appropriate medium. Conveniently, a buffer of appropriate charge, osmolarity, etc. may be added to the sample prior to, simultaneously with, or after contact of the sample with the solid support.

U.S. Pat. No. 6,617,105 discloses a method of isolating nucleic acid from a sample of cells which includes: binding cells in the sample to a solid support coated with cell-binding moieties; and lysing the isolated cells. In this method, a glycidyl-histidine modified magnetic bead is used. However, this method does not disclose a hydrophobic solid support having a specific water contact angle, as used in the present invention.

International Publication No. WO 03/102184 A1 discloses a method of isolating nucleic acid from a sample of cells containing target nucleic acid, comprising: contacting a mixture containing the cells and a flocculating agent capable of aggregating the cells with a solid phase capable of binding the cells; separating the aggregated cells from the mixture using the solid phase; and purifying the target nucleic acid from the cells. The flocculating agent is a polyamine or a cationic detergent. In this publication, a magnetic bead is also used as the solid support. However, there is no disclosure of use of a hydrophobic solid support having a specific water contact angle, as used in the present invention.

If the concentration of cells is very low at an early stage of purification of nucleic acid from the cells, the cells must be enriched. Cell enrichment is especially useful for Lab-on-a-chip (LOC) applications since the sample volume used in a miniaturized chip is generally very small.

The inventors of the present invention have discovered that cells in a mixture can be rapidly and efficiently separated by using a hydrophobic solid support having a specific water contact angle and have completed the present invention.

### SUMMARY OF THE INVENTION

The present invention provides a cell separation method using a hydrophobic solid support. The method comprises contacting a solution containing cells with a hydrophobic solid support having a water contact angle between 70 and 90 degrees.

The present invention also provides an apparatus for separating cells from a sample including the cells. The apparatus comprises a hydrophobic solid support having a water contact angle between 70 and 90 degrees.

### BRIEF DESCRIPTION OF THE DRAWINGS

The above and other features and advantages of the present invention will become more apparent by describing in detail exemplary embodiments thereof with reference to the attached drawings in which:

FIG. 1 is a graph illustrating the number of E. coli cells captured at pH 4 or 7 by various solid supports having different water contact angles;

FIG. 2 shows microscopic images of a hydrophilic alumina substrate having a water contact angle of 20 degrees or less, by which no E. coli cells are captured at pH 4 or 7;

FIG. 3 shows microscopic images of E. coli cells captured by the solid support of the present invention in a flow control system;

FIG. 4 shows microscopic images of gram negative bacteria, either Pseudomonas putida or E. coli BL21 cells, captured by the solid support of the present invention in a flow control system;

FIG. 5 shows microscopic images of gram positive bacteria, either Streptococcus mutans or Staphylococcus epidermidis cells, captured by the solid support of the present invention in a flow control system;

FIG. 6 shows microscopic images of Staphylococcus epidermidis, a gram positive bacterium, illustrating that the number of bacteria captured by the solid support of the present invention is increased due to an additive such as alcohol;

FIG. 7 shows microscopic images of E. coli BL21, a gram negative bacterium, illustrating that the number of cells captured by the solid support of the present invention is increased due to an additive such as alcohol;

FIG. 8 presents top views of designed arrangements of diamond-shaped pillars and brick-shaped pillars;

FIG. 9 shows scanning electron microscope (SEM) images of chips having the diamond-shaped pillar or the brick-shaped pillar arrangements manufactured using a self-assembled monolayer (SAM) coating method on a silicon substrate;

FIG. 10 shows a SEM image of a chip having a diamond-shaped pillar arrangement manufactured using SU-8; and

FIG. 11 shows microscopic images of gram positive or negative bacteria captured by a hydrophobic solid support having a three-dimensional structure of the present invention, i.e. a chip having a pillar structure.

### DETAILED DESCRIPTION OF THE INVENTION

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

According to an aspect of the present invention, there is provided a method of separating cells using a hydrophobic solid support, including contacting a solution containing cells with a hydrophobic solid support having a water contact angle between 70 and 90 degrees.

The present invention relates to a method of capturing cells from a sample using a hydrophobic solid substrate. More particularly, the present invention relates to a method of capturing cells from a sample in a certain pH range using a solid substrate having a specific water contact angle. In this method, an additive capable of inducing cell aggregation or precipitation may be added to the cell sample to increase the extent of capture of the target cells.

One of the methods of cell capture and separation applicable to a miniaturized chip is to use a solid substrate. When a solid substrate is used in an implementation of a LOC, cells can be captured by a physical interaction between cells and the solid substrate. The interaction can be, for example, an electrostatic adsorption or a hydrophobic interaction. Electrostatic adsorption can be used to separate cells by using a material such as alumina which has an electrostatic positive charge because generally the surface of cell is negatively charged. Cell capture based on a hydrophobic interaction with a solid support is a cell attachment resulting from physical interaction originating from the surface free energy between the cells and the solid support, which is governed by surface thermodynamics. Because the hydrophobicity of the surfaces of a cell and the solid support might be dependent on the pH of the surrounding environment, the pH of the sample may be an important factor. A typical method of identifying the hydrophobicity of the solid support surface is to measure a water contact angle. As the water contact angle of the solid surface increases, the hydrophobicity increases.

When a solution including cells is in contact with a hydrophobic solid support having a water contact angle between 70 and 90 degrees, the cells bind to the solid support due to hydrophobic interaction with the hydrophobic solid support. Cells rarely bind to a hydrophilic solid support as demonstrated in Examples below. In addition, when the water contact angle of a hydrophobic solid support does not lie within the above-described range, the degree of binding of cells to the hydrophobic solid support decreases.

The contact of cells with the hydrophobic solid support can be carried out at a range of pH 2.5 to 7, preferably pH 2.5 to 4. The binding efficiency of cells to the hydrophobic solid support is significantly reduced when the pH of a solution including cells does not lie within the above-described range.

In an embodiment of the present invention, the method may further include washing the hydrophobic solid support having cells bound thereto. When the hydrophobic solid support having cells bound thereto is washed with a washing solution such as a phosphate buffered solution, things that are not bound to the hydrophobic solid support are removed while bound cells are retained. Thus, cells can be selectively separated from a mixture containing the cells. Thus, cells can be concentrated.

In the present invention, the solution containing cells can be saliva, blood, urine, buffer or a combination thereof, but is not limited thereto.

In the method, contact of a solution containing cells with a hydrophobic solid support can be carried out in a static state or in a flowing system.

The hydrophobic solid support may have a planar or a three dimensional (3-D) structure. Examples of three-dimensional structures for the hydrophobic solid support are a pillar post, a bead, or a sieve. In a flow control system, 3-D structures enable capture of more cells by increasing the collision rate of the cells with the hydrophobic solid support.

The aspect ratio of a pillar post may be a range of 1:1 to 20:1. The aspect ratio refers to a ratio of the height to the width of a pillar post. When the aspect ratio does not lie within the above-described range, the binding efficiency of cells to the hydrophobic solid support decreases.

The ratio of the height of a pillar post (H) to the distances between pillar posts may be a range of 1:1 to 25:1. When the ratio is less than 1:1, the efficiency of capturing cells decreases.

The distance (D) between pillars may range from 5 to 100 μm. When the distance (D) between pillars is less than 5 μm, it is difficult to fabricate them on a solid support such as a silicon surface. When the distance (D) between pillars is greater than 100 μm, the number of pillars on the same area decreases, and thus the efficiency of capturing cells decreases.

The hydrophobic solid support may be any support capable of capturing cells that does not dissolve in the solution containing the cells. The hydrophobic solid support may take one of various forms, such as substrates, particles, sheets, gels, filters, membranes, fibers, capillaries, tubes, plates, or wells.

Conveniently, the support may be made of glass, silica, latex or a polymeric material. Preferred materials are those presenting a high surface area for binding of the cells. Such supports will generally have an irregular surface and may be, for example, porous or particulate. The hydrophobic solid support may comprise a material that can provide the support with a desired hydrophobicity. The material providing the support with the desired hydrophobicity is not particularly defined as long as it can capture cells. For example, the hydrophobic solid support may comprise a material such as octadecyltrichlorosilane (OTS), tridecafluorotetrahydrooctyl trimethoxysilane (DTS), octadecyldimethyl (3-trimethoxysilyl propyl)ammonium chloride (OTC), or polyethyleneiminetrimethoxysilane (PEIM). The above-listed materials are represented by formulae:

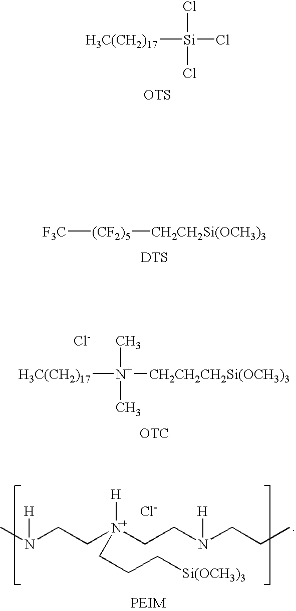

Herein, cells include bacteria (including both gram positive and gram negative bacteria), bacteriophages, plant cells, animal cells, plant viruses and animal viruses. By adding a material capable of inducing cell aggregation and precipitation, such as ethanol or another alcoholic material, the binding efficiency of the cells to the hydrophobic solid support can be further increased. In the method, an alcoholic material capable of inducing cell aggregation and precipitation may be added in the contacting of the solution containing the cells with the hydrophobic solid support. The binding efficiency of cells on the solid support can be increased by aggregating cells or precipitating cells on the solid support using the additive. For example, an increase in the efficiency can be achieved by contacting cells with the support in the presence of alcohol and salt. The use of alcohol and salt in separation and purification procedures such as precipitation is commonplace and any suitable alcohol or salt used in such procedures, may be used according to the present invention. Thus, the alcohol may be any alkanol; specifically lower alkanols such as isopropanol and ethanol are suitable. Other suitable alcohols include methanol and n-butanol.

The salt may be provided by any convenient source, for example, sodium or potassium chloride or acetate, or ammonium acetate. A suitable salt concentration is in a range of 0.01 to 10.0 M, specifically 0.01 to 7.0 M, more specifically 0.01 to 2.0 M. In one embodiment, the salt may be included at the above concentrations in the alcohol solution. Thus, a so-called “cell-binding buffer” may be used containing alcohol and salt at the desired concentrations. Alternatively, the salt and alcohol may be added separately. Appropriate concentrations of alcohol or salt may be determined according to the system and reagents used. Generally, 0.5 to 3 volumes of alcohol to, for example 1 volume of the sample is suitable. Conveniently, the alcohol may be used at concentrations of 50-100% (w/v). The use of alcohol as a precipitant for the cells according to the invention is advantageous for use of the method in clinical diagnostic procedures, since the use of alcohol to conserve clinical samples is common. Thus, patient samples may simply be added to an alcohol-containing cell-binding buffer, whereby the samples are conserved.

As an alternative to precipitation with salt or alcohol, other precipitants may be used, for example, polyethylene glycols (PEGs) or other high molecular weight polymers with similar properties, either alone or in combination with salt and/or alcohol. The concentrations of such polymers may vary depending upon the precise system (i.e., polymer and cell type), but generally concentrations from 1 to 50% (w/v), specifically 2-30% may be used.

As mentioned above, cell binding to hydrophobic solid supports may be achieved by using buffers, often in combination with salt, to achieve pH conditions appropriate for binding. The precise buffers and conditions for cell binding will vary depending on the type of cell, solid support etc.

To allow the cells to bind to the support, various components are mixed and simply allowed to stand for a suitable interval of time. The support may then be removed from the solution, or vice versa, by any convenient means, which will depend, of course, on the nature of the support, and includes all forms of withdrawing the support away from the sample supernatant, or vice versa, for example, centrifugation, pipetting, etc.

The conditions during this process are not critical. The sample can be mixed with the “cell-binding buffer” in the presence of a solid phase and allowed it to stand at room temperature, for example, for 5 to 30 minutes, before separating. As mentioned above, the reaction time is not critical and as little as 5 minutes may often be enough. However, longer periods may be used, for example, 20 minutes to 3 hours, or even overnight. Mixing can be done by any convenient means including stirring or vortexing. Also, if desired, higher or lower temperatures may be used.

Although adsorption of cells to the hydrophobic solid support is preferred according to the invention, it is also possible to use solid supports which have been modified to permit the selective capture of desired cells, for example, cells containing a desired nucleic acid. Thus, for example, supports carrying antibodies, or other binding proteins, for example, lectins, specific for the desired cell type may be used. Thus, such a support may be used to separate and remove the desired target cell type from the sample. The preparation of such selective cell capture matrices is well known in the art and described in the literature.

According to another aspect of the present invention, there is provided an apparatus for separating cells in a sample. The apparatus comprises a hydrophobic solid support having a water contact angle between 70 and 90 degrees. The apparatus can further comprise a chamber for containing a solution containing the cells. The chamber can be a microchamber or a microchannel. The apparatus can further comprise a solution containing the cells.

The present invention will now be described in greater detail with reference to the following examples. The following examples are for illustrative purposes only and are not intended to limit the scope of the invention.

### EXAMPLE 1

### Separation of Escherichia coli Cells Using Hydrophobic Solid Substrate of the Present Invention

To find out whether bacterial cells can efficiently be separated by the method of the present invention, Escherichia coli (E. coli) HB101, a gram negative strain, was used. The substrate used in the experiment was a substrate made of octadecyltrichlorosilane (OTS), tridecafluorotetrahydrooctyl trimethoxysilane (DTS), octadecyldimethyl (3-trimethoxysilyl propyl) ammonium chloride (OTC), or polyethyleneiminetrimethoxysilane (PEIM).

1) Cell Culture

E. coli cells were cultured at 37° C. overnight to exponential phase according to a typical procedure.

2) Preparation of Cell-Containing Solution

E. coli cells cultured overnight were centrifuged at ×800 g for 5 minutes to precipitate the cells. Then, the cells were washed three times with 5 ml of 1× PBS (phosphate buffered saline, pH 7.4, Invitrogen Corporation). Washing was carried out by repeating the process, including resuspending E. coli cells precipitated in 1× PBS and centrifuging the resuspended E. Coli cells at ×800 g for 5 minutes. The washed E. coli cells were suspended in 5 ml of a 0.1 M phosphate buffer (pH 4) or 5 ml of a 0.1 M phosphate buffer (pH 7), respectively. The initial quantity of the suspended E. coli cells was obtained by measuring the optical density (OD) of the suspension using a UV spectrophotometer. Generally, an OD of 1 at 600 nm indicates a concentration of 5.0E+08 cells/ml for E. coli. The E. coli cells suspended in 0.1 M phosphate buffer (pH 4) had an OD at 600 nm of 0.475, corresponding to 1.43E+07 cells for 60 μl, and the E. coli cells suspended in 0.1 M phosphate buffer (pH 7) had an OD at 600 nm of 0.454, corresponding to 1.36E+07 cells in 60 μl.

3) Manufacturing a Solid Support

A solid support was manufactured using a self assembled monolayer (SAM) method. The detailed manufacturing method was as follows.

(1) Washing of Glass Support

Glass supports were immersed in a piranha solution (H₂SO₄:H₂O₂=3:1, 120° C.) for 2 hours or more. Then, the glass supports were spin dried one by one.

(2) Preparation of Coating Solution

OTS or DTS was mixed with toluene such that the final concentration of each mixture was 100 mM. Similarly, OTC or PEIM were mixed with EtOH and stirred for 1 hour.

(3) Immersion of Glass

A glass support prepared above (1) was immersed in a coating solution prepared above (2) for 4 hours.

(4) Washing of Glass

The coated glass support was subsequently washed three times with EtOH, each time for 10 minutes, and then dried in vacuum.

(5) Incubation

The dried coated glass support was incubated at 120° C. for 1 hour.

4) Cell Binding

60 μl Patch (a flexible plastic capable of constituting a chamber on the surface of a solid support and which can be easily adhered to the solid support, for example, due to the presence of glue) was attached to the solid support manufactured above, and then 60 μl of the cell suspension was applied to the solid support. The solid support was incubated at room temperature for 5 minutes, and then washed with 30 ml of a 0.1 M sodium phosphate buffer (pH 4) and 30 ml of a 0.1 M sodium phosphate buffer (pH 7) for each 10 minutes. The washing was carried out in the same manner as described above.

5) Measurement

E. coli cells bound to the solid substrate were stained with a gram stain solution for E. coli cell known in the art. First, a crystal violet solution was sufficiently applied to the region to which cells were bound. After 1 minute, the region was washed with flowing water. Then, the region was successively treated with a gram iodine solution, a gram decolorizer, and a gram safranin solution in the same manner to complete the gram stain. After the gram stain, the solid support was air dried at room temperature. Then, an image of 3 points was captured using an optical microscope at a magnification of ×2000 to determine the number of cells captured per unit area.

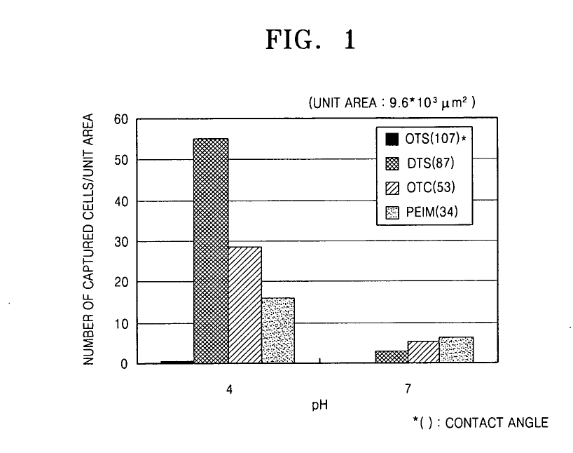

FIG. 1 is a graph illustrating the number of E. coli cells captured at pH 4 or 7 by various solid supports having different water contact angles. The unit area is 9.6×10³ μm² and numerals in parentheses indicate the water contact angle of the solid support. The left panel indicates the number of cells captured at pH 4 and the right panel indicates the number of cells captured at pH 7. Referring to FIG. 1, when the buffer having a pH of 7 was used, E. coli cells rarely bound to the solid support compared to when the buffer having a pH of 4 was used. Thus, it can be seen that pH is important when contacting cells with the hydrophobic solid substrate.

Further, it can be seen that the solid support made with DTS among the 4 types of solid substrate had the highest cell binding efficiency. The solid support made with DTS had a water contact angle of 87 degrees and had higher cell capture efficiency than the other solid supports having different water contact angles, which indicates that the water contact angle is important for cell capture efficiency.

### COMPARATIVE EXAMPLE 1

### Separation of Escherichia coli Cells Using an Alumina Substrate

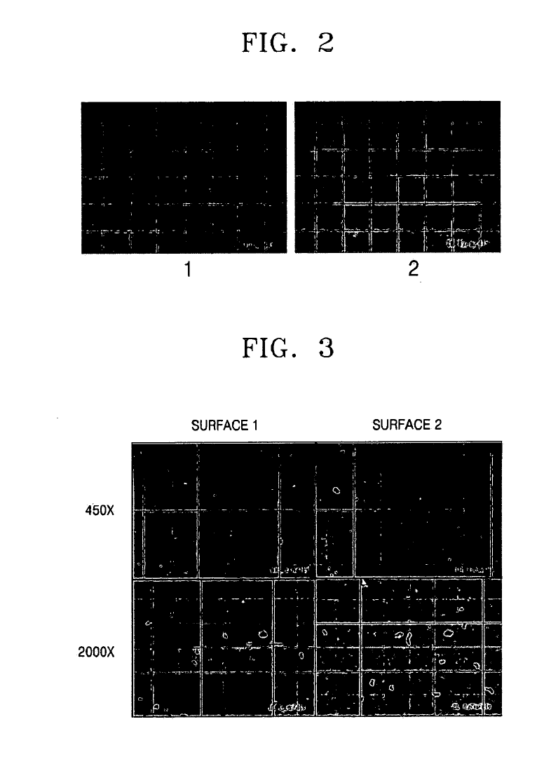

The experiment was carried out in the same manner as in Example 1 except that an alumina substrate having a water contact angle of 14.6 degrees was used instead of the solid supports used in Example 1. FIG. 2 shows microscopic images of the hydrophilic alumina substrate, by which no E. coli cells were captured at pH 4 or pH 7. In FIG. 2, Panel 1 is an image of the alumina substrate at pH 4 and Panel 2 is an image of the alumina substrate at pH 7. Referring to FIG. 2, E. coli cells rarely bound to the substrate at either pH 4 or 7. That is, E. coli cells rarely bound to the hydrophilic alumina having a water contact angle of 30 degrees or less, and thus it can be seen that the water contact angle is an important factor in the binding ability of E. coli cells to a substrate.

### EXAMPLE 2

### Separation of Escherichia coli Cells Under Flow Control

To investigate of the binding ability of E. coli to the substrate of the present invention in a flow control system, a charge reversible surface (CRS) having a water contact angle of 75 degrees (Korean Patent Application No. 2005-0030286, incorporated herein by reference) was used as the surface of a solid support. A CRS is a surface of a solid support for which the surface charge of the surface changes according to the pH of its surroundings. A syringe pump (HARVARD, PHD2000), a flow rate of 0.3 cm/sec (900 μl/min), total surface area of 5 mm×17.3 mm, a cross-sectional area of 5 mm×1.05 mm=5 mm² at an aspect ratio of 5:1 or more, and 200 μl of E. coli HB101 cells in a pH 4 buffer solution (bacterial concentration 1.0E+05 cells/μl) were used. At the flow rate, the 200 μl of bacterial cells were allowed to once pass through the surface area. The substrate was washed by once flowing 1 ml of the sodium phosphate buffer (pH 4, 0.1 M) at the flow rate of 900 μl/min. Subsequent procedures were carried out as in Example 1.

FIG. 3 shows microscopic images of E. coli cells captured by the solid support of the present invention in a flow control system. Surfaces 1 and 2 are the results obtained in duplicate experiments. Referring to FIG. 3, E. coli cells bound to the solid support are not distinguished at a magnification of ×450, but rod-shaped E. coli cells are observed at a magnification of ×2000. Thus, similarly to the fixed system of Example 1, E. coli cells also efficiently bound to the hydrophobic solid support of the present invention in a flow control system.

### EXAMPLE 3

### Separation of Four Different Types of Bacterial Cells Under Flow Control

The experiment was carried out in the same manner as in Example 2 except that the gram negative bacteria E. coli BL21 and Pseudomonas Putida and the gram positive bacteria Streptococcus mutans and Staphylococcus epidermidis were used instead of E. coli HB101.

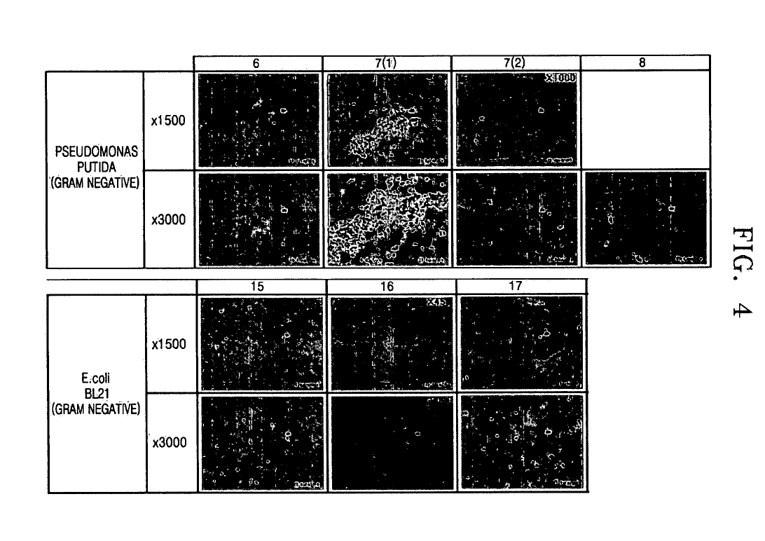

FIG. 4 shows microscopic images of Pseudomonas putida and E. coli BL21 cells captured by the solid support of the present invention in a flow control system. Nos. 6, 7(1), 7(2), 8, 15, 16, and 17 represent Sample Nos. Referring to FIG. 4, Pseudomonas putida and E. coli BL21 cells were relatively uniformly attached to the surface of the solid support. In addition, cell aggregation, as in Sample 7(1), was also observed on some parts of the solid support. Thus, Pseudomonas putida and E. coli BL21 cells efficiently bound to the hydrophobic solid support of the present invention in the flow control system.

The number of Pseudomonas putida and E. coli BL21 cells attached to the hydrophobic solid support of the present invention is summarized in the following Table.

| | Number of attached | | |
|---|---|---|---|
| Bacterial cell | cells | Cells/μm² | Cells/86.5 mm² |
| Pseudomonas | 10.00 | 0.00208 | 1.80E+05 |
| putida | | | |
| E. coli BL21 | 8.40 | 0.00175 | 1.51E+05 |

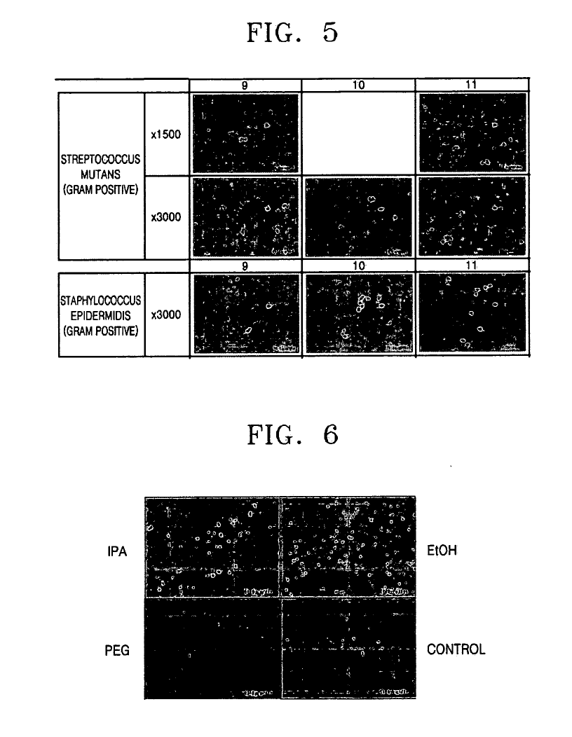

FIG. 5 shows microscopic images of Streptococcus mutans and Staphylococcus epidermidis cells captured by the hydrophobic solid support of the present invention in a flow control system. Nos. 9,10 and 11 represent Sample Nos. of Streptococcus mutans or Staphylococcus epidermidis in the upper and lower panels, respectively. Referring to FIG. 5, Streptococcus mutans and Staphylococcus epidermidis bound to the surface of the hydrophobic solid support of the present invention, but the binding efficiency thereof was much lower than that for the gram negative bacteria shown in FIG. 4.

### EXAMPLE 4

### Separation of Cells Using an Additive

To investigate the effect of the addition of alcohol or salt on the binding ability of cells to the hydrophobic solid support of the present invention in a flow control system, ethyltrimethoxysilane having a water contact angle of 70 degrees was used as the surface of the solid support. A syringe pump (HARVARD, PHD2000), a flow rate of 0.6 cm/sec (900 μl/min), total surface area of 5 mm×17.3 mm, a cross-sectional area of 5 mm×0.5 mm=2.5 mm² at an aspect ratio of 10:1 or more, and 200 μl of bacterial cells (2.0E+07 cells), either E. coli BL21 or Staphylococcus epidermidis, were used.

PEG10000 (w/v 20%) (“PEG” in FIG. 6) in a 0.1 M phosphate buffer (pH 4), EtOH (v/v 50%) (“EtOH” in FIG. 6) in a 0.1 M phosphate buffer (pH 4), or isopropyl alcohol (IPA) (v/v 25%) (“IPA” in FIG. 6) in 3M NaCl (pH 4) were added to the cell suspension medium as additives. The 0.1 M phosphate buffer (pH 4) was used as the control. At the flow rate, 200 μl of bacterial cells were allowed to once pass through the surface area. The hydrophobic solid support was washed while once flowing 1 ml of the sodium phosphate buffer (pH 4) at the flow rate of 900 μl/min. subsequent procedures were carried out as in Example 1.

FIG. 6 shows microscopic images of Staphylococcus epidermidis, a gram positive bacterium, illustrating that the number of bacteria captured by the hydrophobic solid support of the present invention is increased due to the additive such as alcohol. These images are taken at a magnification of ×1000. Referring to FIG. 6, Staphylococcus epidermidis rarely bound to the substrate when the 0.1 M phosphate buffer (pH 4) was used as control. Also, with PEG added to the phosphate buffer, cells rarely bound to the hydrophobic solid support. However, when the additive was IPA or EtOH, a substantial number of cells bound to the hydrophobic solid support. Thus, the number of gram positive cells bound to the hydrophobic solid support of the present invention was small when alcohol and/or salt was absent from the suspending buffer, but was significantly increased when alcohol and/or salt was added.

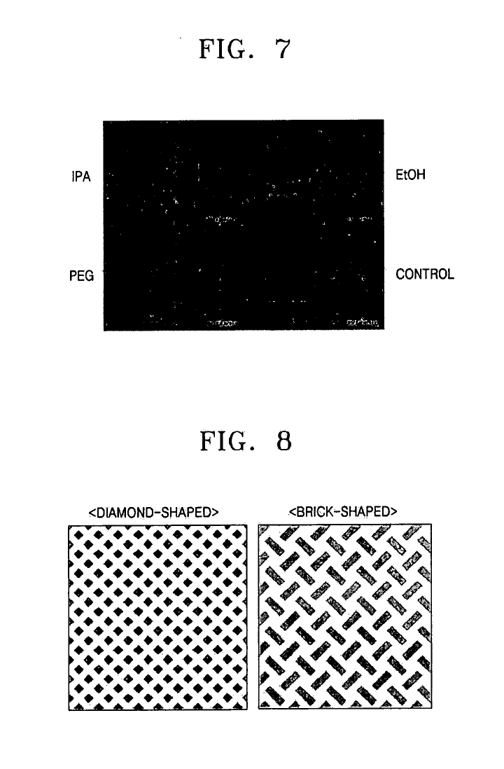

FIG. 7 shows microscopic images of E. coli BL21, a gram negative bacterium, illustrating that the number of cells captured by the hydrophobic solid support of the present invention is increased due to an additive such as alcohol. These images were taken at a magnification of ×1000 and the unit area was 6×10⁴ μm². The number of E. coli BL21 cells bound to the unit area (6×10⁴ μm²) was 33 in the control, 23 in PEG, 123 in IPA, and 145 in EtOH. Referring to FIG. 7, although E. coli BL21 bound to the substrate in 0.1 M phosphate buffer (pH 4) alone, or when PEG was added, more cells bound to the hydrophobic solid support when IPA and EtOH were added to the buffer. Thus, gram negative cells also more easily bind to the hydrophobic solid support of the present invention when alcohol and/or salt are present than when alcohol and/or salt are absent.

### EXAMPLE 5

### Manufacturing Microchip having Hydrophobic Solid Support of a Three-Dimensional Structure

This example investigates the binding ability of E. coli to a microchip of the present invention having a hydrophobic solid support of a three-dimensional structure. Two types of microchips were manufactured.

A silicon-based chip (Si/SiO₂/SAM) was manufactured as follows:

(1) Wafer Cleaning

A wafer was treated in a Piranha solution for 15 minutes, and then washed with flowing water and dried.

(2) Hexamethyldisilazane (HMDS) Coating

Five ml of HMDS was applied to the cleaned wafer using a spin coater; coating was carried out at 500 rpm for 5 seconds and at 4000 rpm for 40 seconds, and then baking was carried out on a hot plate at 120° C. for 2 minutes.

(3) Photoresist (PR) Coating

After applying 5 ml of photoresist (GXR 601) to the wafer, coating was carried out at 500 rpm for 5 seconds and at 4000 rpm for 40 seconds.

(4) Soft Baking

Baking was carried out using a hot plate at 95C for 2 minutes.

(5) UV Exposure

A mask for manufacturing a pillar was mounted on a UV aligner (1-line), and then irradiated at 250 mJ.

(6) Development

Development was carried out using a MIF 300 developing apparatus.

(7) Hard baking

The developed wafer was hard baked at 115° C. for 2 minutes.

(8) Deep RIE

A Si etching process of 100 μm was carried out using STS ICP-RIE apparatus.

(9) Ashing

The photoresist was ashed using an asher.

(10) PR strip

To remove residue PR, the wafer was treated with a Piranha solution for 15 minutes, rinsed, and dried.

(11) HF treatment

The wafer was treated with diluted HF for 1 minute to remove native oxide.

(12) Si oxidation

Thermal wet oxidation was carried out using water vapor to grow SiO₂ to a thickness of 1000 Å.

(13) Wafer Cleaning

The wafer was treated with a Piranha solution for 15 minutes, and then rinsed and dried.

(14) SAM Coating

The wafer was immersed in a 200 mM octadecyldimethyl (3-trimethoxysilyl propyl) ammonium chloride (OTC) in ethanol for 1 hour, and then washed three times with ethanol and dried. A baking process was carried out at 110° C. for 40 minutes.

Next, a SU-8 chip was manufactured as follows:

(1) Wafer Cleaning

The wafer was treated with a Piranha solution for 15 minutes and with diluted HF for 3 minutes, and then rinsed and dried. The wafer was then treated on a hot plate at 200° C. for 20 minutes.

(2) SU-8 Coating

8 ml of SU8 2100 (Microchem) was poured to the wafer, and then spin coating was carried out at 500 rpm for 30 seconds and 3000 rpm for 30 seconds.

(3) Soft Baking

The SU8-coated wafer was treated on a hot plate at 65° C. for 5 minutes and at 95° C. for 20 minutes.

(4) UV Exposure

A mask for manufacturing a pillar was mounted on a UV aligner (1-line), and then irradiated at 320 mJ.

(5) Post Exposure Baking

The wafer was treated at 65° C. for 1 minute and at 95° C. for 20 minutes.

(6) Development

Development was carried out using a SU8 developing apparatus (Microchem) for 30 minutes.

(7) Washing

The wafer was simply washed with 2-propanol and dried with nitrogen.

The three-dimensional structure of the chip was a pillar structure, the chamber volume was 10, the pillar height was 100 μm. Four types of diamond-shaped pillars and 2 types of brick-shaped pillars were manufactured as summarized in the following table.

| | | | | Distance | | | Volume |
|---|---|---|---|---|---|---|---|
| No. of | | Height | Size of | between | | | of |
| silicon-based | | of pillar | pillar | pillars | Aspect | Size of chip | chamber |
| chip | Type of pillar | (μm) | (μm) | (μm) | ratio | (mm) | (μl) |
| 1 | Diamond-shaped | 100 | 25 × 25 | 25 | 4 | 33.89 × 9.89 | 10 |
| 2 | Diamond-shaped | 100 | 50 × 50 | 50 | 2 | 33.89 × 9.89 | 10 |
| 3 | Diamond-shaped | 100 | 25 × 25 | 8 | 4 | 33.89 × 9.89 | 10 |
| 4 | Diamond-shaped | 100 | 50 × 50 | 17 | 2 | 33.89 × 9.89 | 10 |
| 9 | Brick-shaped | 100 | 25 × 75 | 25 | 4 | 33.89 × 9.89 | 10 |
| 10 | Brick-shaped | 100 | 25 × 50 | 12.5 | 4 | 33.89 × 9.89 | 10 |

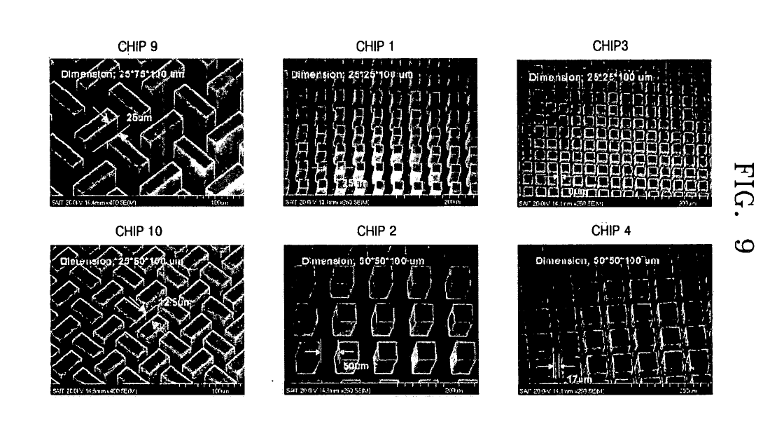

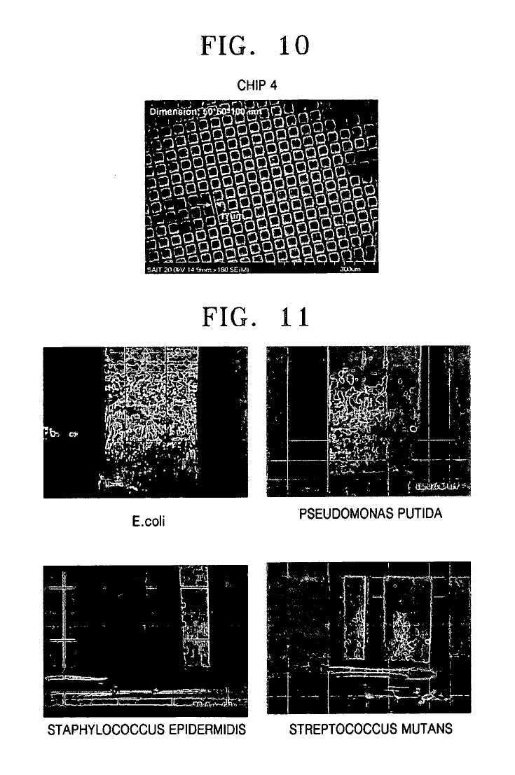

FIG. 8 shows top plan views of designed arrangements of diamond-shaped pillars or brick-shaped pillars. Such chips were manufactured and observed with a scanning electron microscope (SEM). FIG. 9 shows SEM images of chips having diamond-shaped pillar and brick-shaped pillar arrangements manufactured using a SAM method on a silicon substrate. Referring to FIG. 9, it can be seen that diamond-shaped chips and brick-shaped chips having the desired dimensions were manufactured. FIG. 10 shows a SEM image of a chip having a diamond-shaped pillar arrangement manufactured using SU-8. Referring to FIG. 10, it can be seen that a diamond-shaped chip having the desired dimensions was manufactured.

### EXAMPLE 6

### Separation of E. coli Cells Using Three-Dimensional Microchip

As a contrast to Example 2, which used a planar solid substrate, the three-dimensional microchips manufactured in Example 5 were used to investigate the binding ability of E. coli to the microchip of the present invention in a flow control system. E. coli BL21 at 1.0E+08 cells/ml was used with a flow rate of 400 μl/min. The number of E. coli cells bound to the microchip was determined using a method of measuring the number of colony. The subsequent procedures were carried out in the same manner as in Example 2.

The capture efficiency for E. coli cells according to the type of chip is shown in Table 1. “Plane” indicates the case where the planar hydrophobic solid support of Example 2 was used.

TABLE 1

| | | Dis- | | | | |
|---|---|---|---|---|---|---|
| | | tance | Total | Increase | Ratio of | |
| | Pillar | between | number | in | surface | Capture |
| Type of | size | pillars | of | surface | to | efficiency |
| chip | (μm) | (μm) | pillar | area | volume | (%) |
| Chip 1 | 25 × 25 | 25 | 48422 | 6.6 | 0.063 | 91.69 ± 7.1 |
| Chip 2 | 50 × 50 | 50 | 12106 | 3.8 | 0.037 | 24.7 |
| Chip 3 | 25 × 25 | 8 | 181764 | 21.9 | 0.226 | 97.0 ± 2.5 |
| Chip 4 | 50 × 50 | 17 | 42613 | 10.8 | 0.111 | 90.1 ± 3.7 |
| Chip 9 | 25 × 75 | 25 | 23706 | 6.6 | 0.063 | 52.8 ± 7.6 |
| Chip 10 | 25 × 50 | 12.5 | 64155 | 13.1 | 0.140 | 83.3 ± 12.7 |
| Plane | — | — | — | — | 0.002 | ˜1 |

As is apparent from the above Table, Chip 3 having the greatest increase in surface area of pillar had the highest E. coli capture efficiency, roughly two orders of magnitude higher E. coli capture efficiency than when a planar surface was used (capture efficiency of about 1%). Thus, a chip having a pillar structure of the present invention increases opportunity of contact with E. coli, and thus can more efficiently capture E. coli than a planar structure.

### EXAMPLE 7

### Separation of Cells Using a Three-Dimensional Microchip According to Concentration and the Kind of Bacterial Cells

In the present Example, a low concentration of E. coli or other bacterial cells were used to investigate cell capture efficiency of the three-dimensional microchip of the present invention. The experiment was carried out in the same manner as in Example 6, except that E. coli BL21 at 1.0E+03 cells/ml or 1.0E+05 cells/ml was used. Additional bacterial strains, Pseudomonas putida, Staphylococcus epidermidis and Streptococcus mutans, each at 1.0E+08 cells/ml, were used. Chip 10 was used.

The cell capture efficiency according to the kind of bacterial cells is shown in Table 2.

TABLE 2

| | Cell concentration | Capture efficiency |
|---|---|---|
| Type of cell | (cells/ml) | (%) |
| E. coli | 1 × 10³ | 56.2 ± 14 |
| | 1 × 10⁵ | 56.2 ± 6.3 |
| Pseudomonas putida | 1 × 10⁸ | — |
| Staphylococcus | 1 × 10⁸ | 76.2 ± 11.5 |
| epidermidis | | |
| Streptococcus mutans | 1 × 10⁸ | 79.6 ± 2.7 |

In Table 2, the capture efficiency for Pseudomonas putida is not shown. A capture efficiency for Pseudomonas putida could not be quantified by colony counting since the strain did not grow at pH 4, the solution condition for binding of the cells. However, capture of Pseudomonas putida cells was identified through a microscopic image analysis.

As is apparent from the Table 2, the capture efficiency for E. coli BL21 was high at a low concentration and the capture efficiency for bacterial cells other than E. coli was also very high. Thus, a chip having a pillar structure of the present invention can efficiently capture most bacterial cells.

To observe bacteria captured by the chip of the present invention, the captured bacterial cells were stained according to a common staining procedure and observed through an electron microscope. FIG. 11 shows microscopic images of gram positive and negative bacteria captured by a chip having a pillar structure, which is the hydrophobic solid support having a three-dimensional structure of the present invention. Referring to FIG. 11, it can be seen that bacterial cells were efficiently captured by pillars.

### EXAMPLE 8

### Separation of E. coli Cells Included in a Saliva Sample Using Three-Dimensional Microchip

In the present Example, a suspension of bacterial cells in a saliva sample was prepared to investigate the capture efficiency for bacterial cells included in a real sample. E. coli BL21 stained with a SYTO-9 fluorescent dye was used as the bacterial cell. The saliva sample was pre-treated with 0.01 M dithiothreitol (DTT) for 15 minutes or more and centrifuged, followed by resuspending the resultant in a 0.1 M sodium phosphate buffer (pH 4) to dilute to ¼ of the concentration of the initial saliva sample. The pre-treated saliva solution was suspended with the SYTO-9 stained bacteria. The flow rate was 400 μl/min and capture of bacteria was determined using a fluorometer. Subsequent procedures were carried out in the same manner as in Example 6.

The capture efficiency for E. coli cells included in the real sample is shown in Table 3.

TABLE 3

| | Cell concentration | Capture efficiency |
|---|---|---|
| Type of chip | (cells/ml) | (%) |
| Chip 4 (Si/SiO₂/SAM chip) | 5 × 10⁶ | 99.6 ± 0.1 |
| SU-8 chip (the same | 2 × 10⁷ | 98.4 ± 0.3 |
| design as Chip 4, except | | |
| for material | | |

As is apparent from Table 3, both the Si/SiO₂/SAM chip and the SU-8 chip had very high capture efficiency for bacterial cells. Thus, even when a very small amount of bacterial cells is included in a real sample, the bacterial cells can be efficiently captured.

### EXAMPLE 9

### Effect of Buffer pH on Cell Capture Efficiency in Three-Dimensional Structure

A plane structure exhibited the highest cell capture efficiency at a pH of about 4 as shown in Example 1. The effect of pH of 100 mM sodium phosphate on cell capture efficiency of the three-dimensional microchip 3 manufactured in Example 6 was investigated. E. coli was suspended in sodium phosphate having pH 4 or pH 7, respectively, and cell capture efficiency was compared. The experiment was repeated three times while flowing buffers at a rate of 300 μl/min. The effect of pH was significantly offset compared with the plane structure due to the effect of structure. That is, the method of the present invention can also be used over a broader range of pH with a 3-D hydrophobic solid support.

| | Sodium phosphate | Sodium phosphate |
|---|---|---|
| | pH 4 | pH 7 |
| 1 | 98.1% | 92.0% |
| 2 | 98.1% | 88.2% |
| 3 | 98.0% | 91.0% |
| Average | 98.0% | 90.4% |

### EXAMPLE 10

### Separation of Cells Contained in Urine Using Beads

SAM coating was performed on glass beads using octadecyldimethyl (3-trimethoxysilyl propyl) ammonium chloride (OTC) as in Example 6. In contrast to Example 8 in which the saliva was pretreated, urine was taken without any pre-treatment process and mixed with an E. coli solution of a known concentration (5×10⁶ cells/ml, sodium phosphate 100 mM, pH 3) at a ratio of 1:1. 0.2 g of OTC-coated beads were added to the sample and mixed. After separation of the beads from the sample, cells adsorbed by the beads were quantified using a colony count method. The beads and the sample were mixed for 1, 15, and 30 minutes. While saliva required a pre-treatment process, urine exhibited a bacterial cell capture efficiency of about 50% after only 15 minutes without any pre-treatment process.

| Mixing time | Cell concentration | Capture efficiency |
|---|---|---|
| (min) | (cells/ml) | (%) |
| 1 | 5 × 10⁶ | 30 |
| 15 | 5 × 10⁶ | 50 |
| 30 | 5 × 10⁶ | 50 |

As described above, by simply allowing cells to be adsorbed to a hydrophobic solid substrate, cells can be separated. Thus, cell separation efficiency can be rapidly and simply increased. Further, the cell separation efficiency can be significantly improved using a three-dimensional microstructure instead of a planar, two-dimensional structure. Cell separation is possible in a flow control system within several minutes. Moreover, cell separation from a sample such as saliva or urine is possible and the present invention can be applied to the preparation of a sample in a LOC.

Any combination of the above-described elements in all possible variations thereof is encompassed by the invention unless otherwise indicated herein or otherwise clearly contradicted by context.

1. A method of separating cells using a hydrophobic solid support, comprising
contacting a solution containing cells with a hydrophobic solid support having a water contact angle between 70 and 90 degrees.

2. The method of claim 1, wherein the contacting is carried out in a static or fluidic state.

3. The method of claim 1, wherein the pH of the solution containing cells is in the range of 2.5 to 7.

4. The method of claim 3, wherein the pH of the solution containing cells is in the range of 2.5 to 4.

5. The method of claim 1 or 3, wherein the solution containing cells is saliva, blood, urine, buffer, or a combination thereof.

6. The method of claim 1, wherein the hydrophobic solid support has a planar structure, a pillar structure, a bead structure, or a sieve structure.

7. The method of claim 6, wherein an aspect ratio of the pillar has a range of 1:1 to 20:1.

8. The method of claim 6, wherein the ratio of a pillar height (H) to a distance (D) between pillars has a range of 1:1 to 25:1.

9. The method of claim 6, wherein the distance (D) between pillars has a range of 5 to 100 μm.

10. The method of claim 1, wherein the cells are selected from the group consisting of bacteria, bacteriophages, plant cells, animal cells, plant viruses and animal viruses.

11. The method of claim 1, wherein in the contacting, salt or alcohol is further added to the solution of cells.

12. The method of claim 11, wherein the concentration of the salt has a range of 0.01 to 2 M.

13. The method of claim 11, wherein the alcohol is selected from the group consisting of ethanol, isopropanol, methanol, and n-butanol.

14. The method of claim 1, further comprising
washing the hydrophobic solid support having the cells bound thereto.

15. An apparatus for separating cells in a sample, comprising
a hydrophobic solid support having a water contact angle between 70 and 90 degrees.

16. The apparatus of claim 15, wherein the hydrophobic solid support has a planar structure, a pillar structure, a bead structure, or a sieve structure.

17. The apparatus of claim 16, wherein an aspect ratio of the pillar has a range of 1:1 to 20:1.

18. The apparatus of claim 16, wherein the ratio of a pillar height (H) to a distance (D) between pillars has a range of 1:1 to 25:1.

19. The apparatus of claim 16, wherein the distance (D) between pillars has a range of 5 to 100 μm.

20. The apparatus of claim 15, further comprising a sample chamber.
